(19) United States
(12) Patent Application Publication
(10) Pub. No.: US 2015/0164373 A1
(43) Pub. Date: Jun. 18, 2015

(54) PERSONAL LUNG FUNCTION MONITORING DEVICE CAPABLE OF EXHALED BREATH ANALYSIS

(71) Applicant: The Regents of the University of California, Oakland, CA (US)

(72) Inventors: Cristina E. Davis, Davis, CA (US); Jean-Pierre Delplanque, Davis, CA (US); Nicholas J. Kenyon, Sacramento, CA (US); Alice M. Kwan, Davis, CA (US)

(21) Appl. No.: 14/407,225

(22) PCT Filed: Jun. 11, 2013
(86) PCT No.: PCT/US2013/045259

§ 371 Date: Dec. 11, 2014

Related U.S. Application Data
(60) Provisional application No. 61/659,271, filed on Jun. 13, 2012.

(51) Int. Cl. A61B 5/08; A61B 5/00; A61B 5/087

(52) U.S. Cl. CPC A61B 5/0803; A61B 5/082; A61B 5/0871; A61B 5/0002

(57) ABSTRACT

A personal lung function monitoring device capable of exhaled breath analysis is described. The personal lung function monitoring device includes a physical measuring device and a microcontroller. This physical measuring device further includes a flow chamber configured to receive a flow of exhaled breath from a patient/user, and a set of sensors integrated with the flow chamber. The set of sensors can be used to measure a set of properties of the exhaled breath, which can include one or more common lung function parameters and/or one or more biomarkers of the exhaled breath. The microcontroller is coupled to the physical measuring device and configured to receive analog sensor signals from the set of sensors and transmit the digitized sensor signals to a mobile device. In one embodiment, the personal lung function monitoring device combines peak expiratory flow, spirometry, and exhaled breath biomarker measurements into a single device.

### BACKGROUND

1. Field

The present disclosure generally relates to a personal lung function monitoring device. More specifically, the present disclosure relates to the design and operation of a portable personal lung function monitoring device coupled with a mobile device that allows for transmitting the measured physiological and biochemical data via telemetry.

2. Related Art

Exhaled human and animal breath is thought to contain “fingerprints” of disease/disorder related chemical compounds. Hence, exhaled breath analysis has become increasingly of interest as a diagnostic tool, for example, for various diseases, including: asthma, cancer, and respiratory infections.

In particular, asthma affects over 300 million people worldwide. Those afflicted with asthma have difficulty breathing and experience airflow obstruction caused by inflammation and constriction of the airways. The severity of asthma varies, and existing therapy and medication regimens only help to alleviate the symptoms temporarily. Home monitoring of lung function and asthma symptoms is the recommended course of action to give physicians and asthma patients a chance to control the disease jointly. To advance this field, it is necessary to develop accurate and efficient lung function monitoring techniques that allow patients to easily monitor common lung function parameters, such as spirometry and peak expiratory flow (PEF).

Unfortunately, each lung function test is currently performed separately on specialized devices and/or machines, which precludes data comparison and complicates longitudinal health assessment outside of the hospital setting. While classic spirometry is currently the preferred method to capture a complete picture of airflow obstruction and lung function, the existing spirometers are often bulky and generally require supervision. Portable peak flow meters are presently available but are usually imprecise and inconvenient to use. Moreover, there are no portable lung function monitoring devices commercially available that can simultaneously perform exhaled breath analysis.

Hence, what is needed is a personal lung function monitoring device without the problems described above.

### SUMMARY

One embodiment of the present disclosure provides a personal lung function monitoring device capable of exhaled breath analysis. The personal lung function monitoring device includes a physical measuring device and a microcontroller. This physical measuring device further includes a flow chamber configured to receive a flow of exhaled breath from a patient/user, and a set of sensors integrated with the flow chamber. The set of sensors can be used to measure a set of properties of the exhaled breath, which can include one or more common lung function parameters and/or one or more biomarkers of the exhaled breath. The microcontroller is coupled to the physical measuring device and configured to receive analog sensor signals from the set of sensors and transmit the digitized sensor signals to a mobile device, for example, through a USB connection. In one embodiment, the personal lung function monitoring device combines peak expiratory flow, spirometry, and exhaled breath biomarker measurements into a single device.

In some embodiments, the set of properties of the exhaled breath includes one or more common lung function parameters and/or one or more biomarkers of the exhaled breath.

In some embodiments, the set of sensors includes at least one sensor to measure a common lung function parameter and at least one sensor to measure a biomarker of the exhaled breath.

In some embodiments, the set of sensors includes at least one flow sensor and at least one chemical sensor.

In some embodiments, the set of sensors includes one or more sensors configured to perform a spirometry test on the exhaled breath.

In some embodiments, the set of sensors includes one or more sensors configured to perform a peak expiratory flow (PEF) measurement on the exhaled breath.

In some embodiments, the set of sensors includes two or more chemical sensors configured to measure two or more biomarkers of the exhaled breath.

In some embodiments, the two or more biomarkers include two or more of the following: nitric oxide (NO); carbon monoxide (CO); oxygen (O₂); and other chemical biomarkers.

In some embodiments, the set of sensors includes: one or more sensors for spirometry tests of the exhaled breath; one or more sensors for PEF measurements of the exhaled breath; and one or more sensors for biomarker measurements of the exhaled breath.

In some embodiments, the set of sensors is arranged linearly along the path of the exhaled breath through the flow chamber.

In some embodiments, the flow chamber includes a cylindrical tube which has a first end and a second end. The exhaled breath enters the cylindrical tube from the first end and exits the cylindrical tube from the second end. The wall of the cylindrical tube includes a set of openings configured to receive the set of sensors.

In some embodiments, a given opening in the set of openings is configured so that the size and shape of the given opening match the size and shape of a given sensor in the set of sensors, which is inserted into the given opening.

In some embodiments, the cylindrical tube is partitioned into a first section and a second section by an orifice plate placed in the path of the exhaled breath, wherein the hole of the orifice plate has a diameter that is smaller than the diameter of the cylindrical tube.

In some embodiments, the set of sensors includes a differential pressure sensor attached to two pressure taps: the first pressure tap is positioned in the first section before the orifice plate, and the second pressure tap is positioned in the second section of the cylindrical tube after the orifice plate.

In some embodiments, two differential pressure sensors are used cooperatively to perform a spirometry test.

In some embodiments, the set of sensors further includes a set of chemical sensors, which is positioned in the second section of the cylindrical tube and is further away from the orifice plate than the second pressure tap.

In some embodiments, the microcontroller includes an analog-to-digital converter (ADC) configured to digitize the received analog sensor signals.

In some embodiments, the physical measuring device further comprises sensor circuits associated with the set of sensors.

In some embodiments, the microcontroller is coupled to the physical measuring device through the sensor circuits by a stacked-circuit-board structure.

In some embodiments, the microcontroller is coupled to the mobile device through a USB connection.

Another embodiment of the present disclosure provides a system for performing a lung function test using a personal lung function monitoring device. During operation, a physical measuring device of the personal lung function monitoring device receives exhaled breath from a user. The physical measuring device further includes a flow chamber configured to receive the exhaled breath, and a set of sensors integrated with the flow chamber and configured to measure a set of properties of the exhaled breath, which can include at least one common lung function parameter and at least one biomarker of the exhaled breath. Next, a microcontroller coupled to the physical measuring device processes analog sensor signals received from the set of sensors. The microcontroller subsequently transmits the processed sensor signals to a mobile device coupled with the personal lung function monitoring device.

### BRIEF DESCRIPTION OF THE FIGURES

FIG. 1 presents a block diagram illustrating a personal lung function monitoring device in accordance with an embodiment of the present disclosure.

FIG. 2A illustrates an exemplary design of the flow chamber of the physical measuring device in FIG. 1 in accordance with an embodiment of the present disclosure.

FIG. 2B illustrates a cutaway view of the flow tube in FIG. 2A in accordance with an embodiment of the present disclosure.

FIG. 3 illustrates a cutaway view of an exemplary design of the physical measuring device in FIG. 1 in accordance with an embodiment of the present disclosure.

FIG. 4 illustrates a cutaway view of a physical measuring device, which is obtained by integrating physical measuring device 300 in FIG. 3 with an orifice plate in accordance with an embodiment of the present disclosure.

FIG. 5A illustrates an exemplary design of the portable device and the associated microcontroller in FIG. 1 in accordance with an embodiment of the present disclosure.

FIG. 5B illustrates the coupling between portable device 500 in FIG. 5A and a mobile device in accordance with an embodiment of the present disclosure.

FIG. 6 presents a flowchart illustrating a process for using the portable device to monitor common lung function parameters and/or biomarkers of the exhaled breath in accordance with an embodiment of the present disclosure.

Note that like reference numerals refer to corresponding parts throughout the drawings. Moreover, multiple instances of the same part are designated by a common prefix separated from an instance number by a dash.

### DETAILED DESCRIPTION

Embodiments of a personal lung function monitoring device that includes a physical measuring device and a microcontroller, and a method for controlling breath maneuvers using the personal lung function monitoring device are described. This physical measuring device includes a flow chamber configured to receive a flow of exhaled breath from a patient/user, and a set of sensors integrated with the flow chamber. The set of sensors can be used both to measure common lung function parameters and to perform exhaled breath analysis. The microcontroller is coupled to the physical measuring device and configured to receive analog sensor signals from the set of sensors and transmit the digitized sensor signals to a mobile device, for example, through a USB connection. In one embodiment, the personal lung function monitoring device combines peak expiratory flow, spirometry, and exhaled breath biomarker measurements into a single device.

In some embodiments, the set of sensors includes at least one sensor to measure a common lung function parameter and at least one sensor to measure a chemical biomarker (or simply “biomarker”) of the exhaled breath.

Moreover, the set of sensors may include one or more sensors configured to perform spirometry measurements of the exhaled breath; one or more sensors configured to perform a peak expiratory flow (PEF) measurements of the exhaled breath; and one or more chemical sensors configured to measure a set of chemical biomarkers of the exhaled breath. In one embodiment, the flow chamber includes a cylindrical tube wherein the wall of the cylindrical tube includes a set of openings for receiving the set of sensors. In some embodiments, a given opening in the set of openings is configured so that the size and shape of the given opening match the size and shape of a given sensor in the set of sensors, which is inserted into the given opening.

A compact personal lung function monitoring device provides the capability of monitoring a set of important lung function parameters and simultaneously measuring a set of biomarkers of the exhaled breath, and subsequently recording the data to a personal mobile device, such as a smartphone. The proposed personal lung function monitoring device allows patients to monitor common lung function parameters (e.g., peak expiratory flow (PEF), spirometry) and biomarkers of the exhaled breath using their personal mobile device at any time and at any location. The disclosure relates to design of such a personal lung function monitoring device that combines PEF, spirometry, and exhaled breath biomarker measurements into a single device, making lung function monitoring less time-consuming and easier to obtain.

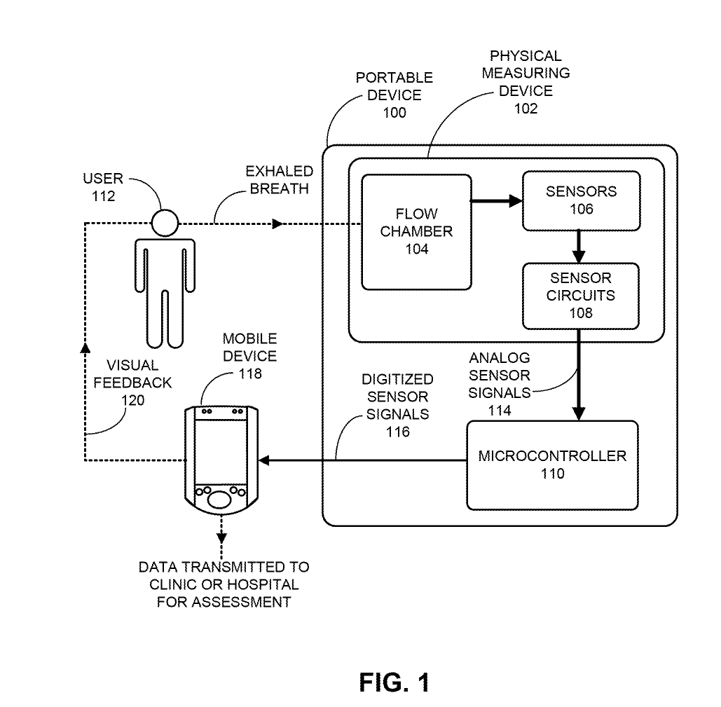

We now describe embodiments of a personal lung function monitoring device. FIG. 1 presents a block diagram illustrating a personal lung function monitoring device 100 in accordance with an embodiment of the present disclosure. As can be seen in FIG. 1, personal lung function monitoring device 100 (also referred to as “portable device 100” hereinafter) includes: a physical measuring device 102 which further comprises a flow chamber 104, a set of sensors 106, and associated sensor circuits 108; and a microcontroller 110 which is coupled to physical measuring device 102 for data capturing.

Further referring to FIG. 1, to use portable device 100 to measure the flow rates and/or chemical composition of exhaled breath, a user 112, such as an asthma patient, exhales into flow chamber 104 of physical measuring device 102, wherein flow chamber 104 is configured to receive the exhaled breath from user 112 and allow the exhaled breath to travel through flow chamber 104. Flow chamber 104 is also integrated with the set of sensors 106 which is exposed to the inside of the flow chamber 104, so that when the exhaled breath travels through flow chamber 104, the set of sensors 106 is able to capture measurands of the exhaled breath associated with both a set of lung function parameters and a set of biomarkers of the exhaled breath.

Sensor circuits 108 associated with sensors 106 then generate analog sensor signals 114 associated with the set of lung function parameters and/or the set of biomarker concentrations. Analog sensor signals 114 are subsequently received by microcontroller 110, which is configured to process the received analog sensor signals 114 from sensor circuits 108. In some embodiments, microcontroller 110 is configured to amplify the received signals, filter them for noise, and digitize the signals during the acquisition process. Microcontroller 110 subsequently transmits digitized sensor signals 116 to a mobile device 118 using either wired or wireless connections. Note that mobile device 118 can include laptop computers, tablet computers, smartphones, and/or other modern portable computing devices. In one embodiment, microcontroller 110 utilizes a host shield to send digitized sensor signals 116 to mobile device 118 through a standard USB connection.

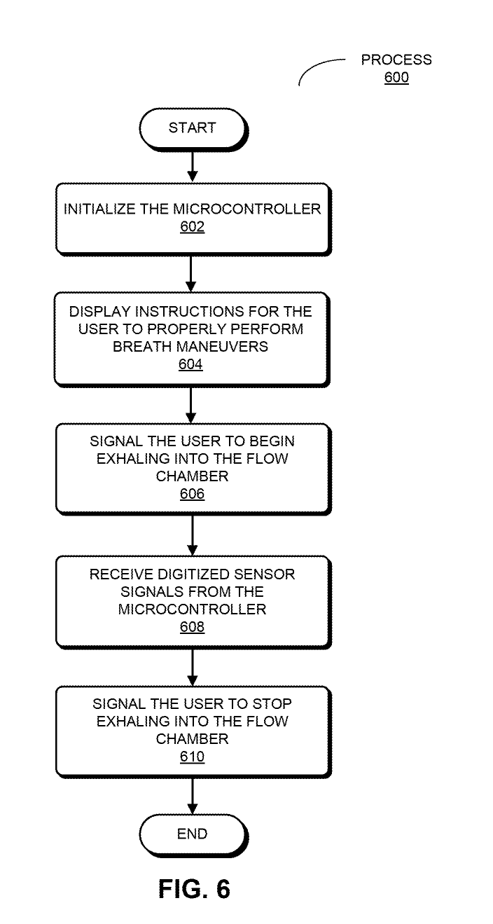

In some embodiments, mobile device 118 uses an installed software application to process digitized sensor signals 116 received from microcontroller 110. The processed sensor signals can be used to generate visual feedback 120, which may include physiologically relevant information to user 112. Mobile device 118 can then display visual feedback 120 to user 112 to guide the user to perform further breath maneuvers. Mobile device 118 can also transmit the processed sensor signals to a clinic or hospital for further assessment via telemetry. Note that the installed software application on mobile device 118 can also be used to guide the entire breath maneuver for user 112 via the UI of mobile device 118. We describe more detailed implementation of the software application on mobile device 118 below in conjunction with FIG. 6.

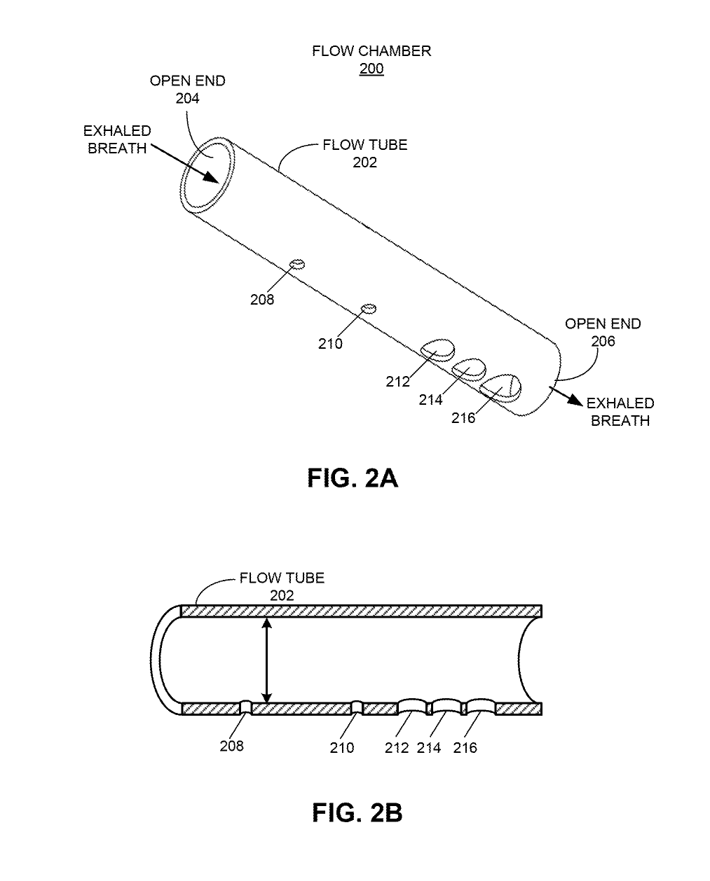

We now describe detailed embodiments of each component of portable device 100. FIG. 2A illustrates an exemplary design of flow chamber 104 of physical measuring device 102 in FIG. 1 (referred to hereafter as flow chamber 200) in accordance with an embodiment of the present disclosure. As can be seen in FIG. 2A, flow chamber 200 includes a flow tube 202 which has a first open end 204 and a second open end 206. Note that while flow tube 202 is shown to have a cylindrical shape, other embodiments of flow chamber 200 may use other tube geometries that have non-circular cross-sections. The exhaled breath from a user/patient enters flow tube 202 from first open end 204, and then travels the length of flow tube 202 wherein the residual of the exhaled breath exits flow tube 202 from second open end 206. In one exemplary design, flow tube 202 has a length of 192 mm, an inner diameter of 28 mm and an outer diameter of 31 mm.

The wall of flow tube 202 includes a set of openings/holes that are configured to receive the set of sensors described in FIG. 1 (sensors not shown). While flow tube 202 is shown to include five openings 208-216 for integrating five sensors, other embodiments of flow chamber 200 can include fewer or additional openings into flow tube 202. Note that the set of openings 208-216 can have different sizes and different shapes. Hence, a given opening may be configured to match the size and shape of a given sensor to be integrated with flow chamber 200.

FIG. 2B illustrates a cutaway view of flow tube 202 in accordance with an embodiment of the present disclosure. While the set of openings 208-216 is shown to have been arranged linearly along the axis of flow tube 202, in other embodiments, a given flow chamber 200 can include a set of openings that is positioned such that at least two openings are not arranged linearly along the axis of flow chamber 200. For example, a pair of sensors, which is used to generate a pair of differential signals, can be placed substantially side by side along the circumference of the flow chamber 200.

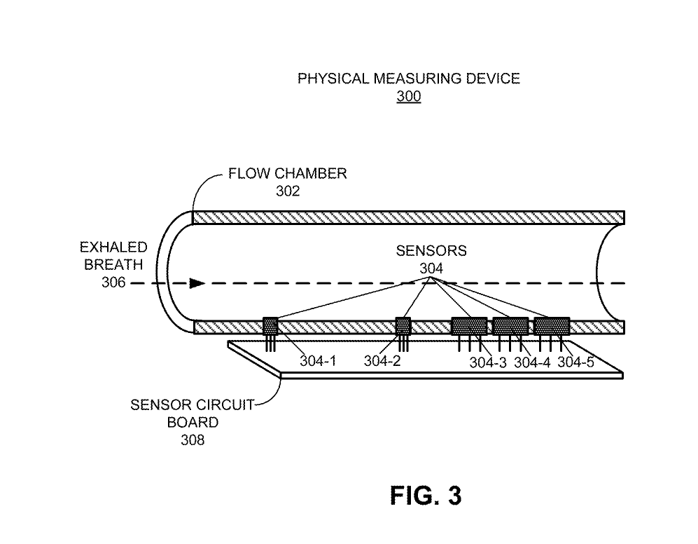

FIG. 3 illustrates a cutaway view of an exemplary design of physical measuring device 102 in FIG. 1 (referred to hereafter as physical measuring device 300) in accordance with an embodiment of the present disclosure. As can be seen in FIG. 3, physical measuring device 300 comprises a flow chamber 302 (which is substantially identical to flow chamber 200 in FIG. 2A) and a set of sensors 304. A flow of exhaled breath 306 enters flow chamber 302 from the left and travels to the right through the flow tube. Flow chamber 302 includes five openings along the tube wall, which are arranged in a linear fashion, and each of the openings is fitted with a sensor among the set of sensors 304.

In the embodiment shown, each of the sensors 304 is positioned vertically such that the top surface of the sensor is either substantially level with, or slightly higher than the inner wall of flow chamber 302. In this manner, the set of sensors 304 does not significantly disrupt the flow of exhaled breath 306 within flow chamber 302. Further illustrated in FIG. 3, the set of sensors 304 is attached to a sensor circuit board 308, which is placed directly underneath flow chamber 302. However, in some embodiments, one or more of the set of sensors 304 can be “flow-through” sensors. In such embodiments, a flow-through sensor is positioned further inside flow chamber 302 through a respective opening, wherein the body of the flow-through sensor includes holes to allow exhaled breath 306 to “flow through” the sensor largely unobstructed.

In one embodiment, the set of sensors 304 includes at least one flow sensor and at least one chemical sensor. The at least one chemical sensor can be used to monitor either a gas phase biomarker or a non-gas phase biomarker concentration in exhaled breath 306. In one embodiment, the set of sensors 304 includes multiple chemical sensors, which are configured to simultaneously measure more than one chemical biomarker concentration. In a further embodiment, the set of sensors 304 includes a single chemical sensor, which is configured to simultaneously measure two or more chemical biomarker concentrations.

In one embodiment, the at least one flow sensor is used for performing automatic spirometry tests. A spirometry test is a physiological test that measures the volume and flow rate of air that can be inhaled and exhaled by a subject. As a lung function monitoring maneuver, it is useful in describing the disease state in the lungs, assessing therapeutic intervention, and/or monitoring for adverse reactions to medication. In one embodiment, the at least one flow sensor can be configured to measure the following spirometry parameters: (1) forced vital capacity (FVC), described as the volume delivered during expiration when made as forcefully and completely as possible starting from full inspiration; (2) the forced expiratory volume in one second (FEV₁), which is the volume delivered in the first second of the FVC maneuver; and (3) a spirometry graph mapping the flow rate (L/s) over exhaled volume (L). Note that it is often beneficial to use a spirometry graph to visualize lung functions in a graphical manner to help physicians evaluate lung health.

In one embodiment, the set of sensors 304 also includes one or more flow sensors for automatically measuring peak expiratory flow (PEF) from exhaled breath 306. A PEF measurement, which measures the maximum flow rate of expiration, correlates to the degree of obstruction in the airways. A PEF measurement is usually gathered within 60 milliseconds of forced exhalation initiation, and is generally considered as an accurate, repeatable, and non-invasive test for monitoring airflow at home.

Note that to fully capture the range of high and low flow rates of exhaled breath, it is necessary to select a cost-effective flow sensor that is also accurate throughout the normal range of the exhaled breath. In the embodiment illustrated in FIG. 3, two flow sensors are employed in physical measuring device 300: flow sensor 304-1 is set up for measuring high flow rates, and pressure sensor 304-2 is set up for measuring low flow rates. During operation, each of the two flow sensors translates the pressure drop from the flow chamber 302 into a voltage signal, which can then be converted to a flow rate after sensor calibration. This two-flow-sensor scheme minimizes the cost of the device, while ensuring the accuracy of the flow measurements throughout a complete spirometry maneuver.

In one embodiment, the set of sensors 304 includes one or more chemical sensors configured to measure a set of biomarkers in exhaled breath 306. In case of a gas phase biomarker, the one or more chemical sensors can be used to monitor one or more of nitric oxide (NO), carbon monoxide (CO), oxygen (O₂), and other gas phase biomarkers in exhaled breath 306. In one embodiment, the three rightmost sensors 304-3, 304-4, and 304-5 in the set of sensors 304 are used to monitor NO, CO, and O₂ concentrations in exhaled breath 306, respectively. For example, three electrochemical sensors (for measuring NO, CO, and O₂ concentrations, respectively) can be selected to detect the lower end of a respective biomarker concentration range found in exhaled breath 306 (e.g., 0.03 ppm for NO, 2 ppm for CO, and 14 pph for O₂). Note that such chemical testing of biomarkers in the exhaled breath provides a non-invasive approach for health care professionals to detect possible signs of inflammation in the airways of patients.

Note that these electrochemical sensors may have to be sufficiently small in size so that they can be integrated with flow chamber 302. In some embodiments, electrochemical sensors having short response times may be preferred because they help to ensure that patients do not tire easily when providing breath samples. In a further embodiment, chemical sensors may be selected for their combination of high sensitivity, high selectivity, miniature size, and short response time compared to other available chemical sensors.

In a particular embodiment, physical measuring device 300 can use the set of sensors 304 to simultaneously perform spirometry (FVC, FEV, FEV₁, and spirometry graph), PEF, and chemical breath biomarker measurements within the same device. Hence, when physical measuring device 300 is used in place of physical measuring device 102 in FIG. 1, portable device 100 allows patients to monitor common lung function parameters (including but not limited to spirometry parameters, PEF, etc.) and biomarkers of the exhaled breath using their personal mobile device at any time and at any location. In one embodiment, all of these lung function measurements can be taken through two breath maneuvers, which are described in more detail below in conjunction with FIG. 6.

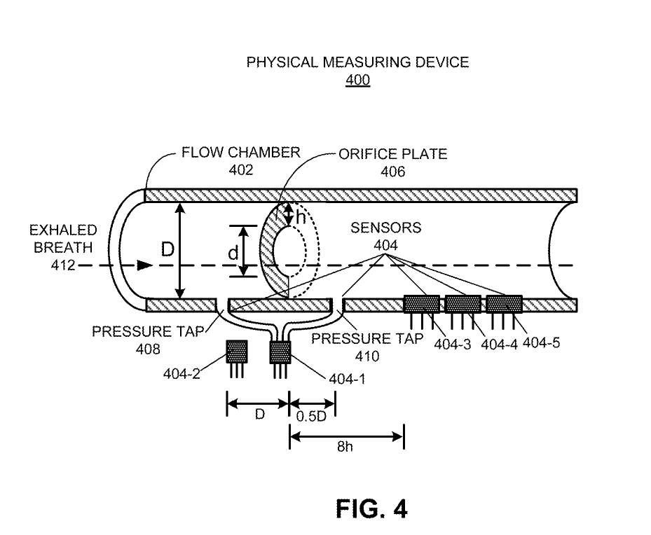

In some embodiments, in order to measure the flow rate of the exhaled breath, a thin orifice plate tailored for the pulmonary flow rates is placed in the path of the exhaled breath, thereby separating the flow tube into two regions. For example, FIG. 4 illustrates a cutaway view of a physical measuring device 400, which is obtained by integrating physical measuring device 300 with an orifice plate 406 in accordance with an embodiment of the present disclosure. More specifically, physical measuring device 400 comprises a flow chamber 402 of inner diameter D, and a set of sensors 404. In particular, sensor 404-1 on the left is a differential pressure sensor connected to two pressure taps (openings) 408 and 410, while sensors 404-3, 404-4, and 404-5 on the right are chemical biomarker sensors. In one embodiment, two differential pressure sensors 404-1 and 404-2 (its connections to pressure taps 408 and 410 not shown) are set up to measure the high flow rates and the low flow rates, respectively. In this embodiment both sensors can be connected to the same pressure taps 408 and 410. In another embodiment, each of the two pressure taps 408 and 410 is configured as two side-by-side openings to receive the two taps of each of the differential pressure sensors 404-1 and 404-2 (i.e., there are a total of 7 openings in the wall of flow chamber 402). A thin orifice plate 406 is placed in the path of exhaled breath 412 between two pressure taps 408 and 410. Note that orifice plate 406 partially obstructs flow chamber 402 and only allows exhaled breath 412 to pass through the orifice of diameter d<D.

Note that the design of physical measuring device 400 forms a simplified obstruction flow meter, which facilitates an instantaneous flow rate measurement in a pipe that follows the Bernoulli obstruction theory (which describes the properties of a flow that is forced by an obstruction from a duct of diameter D into a smaller flow passage of diameter d). Mass conservation dictates that a decrease in the flow area causes an increase in the flow velocity, which is associated with a drop in pressure. A relationship between the pressure drop and volume flow rate is obtained from Bernoulli's equation:

$$\begin{matrix}Q = {C}_{o} {A}_{o} \sqrt{\frac{2 ( {p}_{1} - {p}_{2} )}{ρ ( 1 - {β}^{4} )}} , & ( 1 ) \\ β = \frac{d}{D} , & ( 2 )\end{matrix}$$

where A₀ is the area of the orifice in orifice plate 406, p₁ is the pressure upstream (i.e., to the left) of orifice plate 406, p₂ is the pressure downstream (i.e., to the right) of orifice plate 406, ρ is the density of exhaled breath 412, β is the diameter ratio as shown in Equation 2, and finally Cₒ is the orifice discharge coefficient which is typically on the order of 0.6. Typically, the orifice discharge coefficient is a function of the Reynolds number and β. Note that variable h shown in FIG. 4 is the length of the orifice plate between diameters D and d.

Further referring to FIG. 4, note that the flow of exhaled breath 412 in the region immediately to the right of orifice plate 406 is obstructed and highly non-uniform. Hence, it is necessary that chemical biomarker sensors 404-3 to 404-5 be placed away from orifice plate 406 at a location where the flow is again unobstructed and uniform. However, these sensors may not be placed too far from orifice plate 406 because that would require a long flow tube, which is in conflict with the design requirement of keeping the flow tube as short as possible. Consequently, the placement location of the chemical sensors may be a function of both the flow rate and the diameter of the orifice d. In one embodiment, the chemical sensors may be placed at least 8 times the value of h away from orifice plate 406 to ensure that exhaled breath 412 traveling through flow chamber 402 would flow over the chemical sensors. In an exemplary design, the following parameter values are used: D=28 mm, d=14 mm, h=7 mm; pressure tap 408 is at a distance D to the left of orifice plate 406, pressure tap 410 is at a distance 0.5D to the right of orifice plate 406, and the closest chemical biomarker sensor 404-3 is at a distance 8h away from orifice plate 406. In one embodiment, both flow chamber 402 and the case that houses all the other components of physical measuring device 400 are constructed out of acrylonitrile butadiene styrene (ABS) using a Stratasys FDM rapid prototyping machine (GoEngineer; Santa Clara, Calif.).

In one embodiment, flow chamber 402 is designed to meet the requirements of a thin plate orifice volume flow meter set by the American Society of Mechanical Engineers (ASME). When the criteria are met, the flow meter theoretically operates within a Reynolds number range of 10⁴-10⁷, which indicates that flow chamber 402 operates in the turbulent regime.

In a particular embodiment, two piezoresistive pressure sensors are selected to monitor a broad range of high flows of 50-900 L/min (i.e., pressure sensor 404-1; model #MPX5010; Freescale Semiconductor; San Jose, Calif.), and low flows of 15-100 L/min (i.e., pressure sensor 404-2; model #SSCSNBN002NDAA5; Honeywell; Morristown, N.J.). Furthermore, three commercial-off-the-shelf (COTS) electrochemical chemical sensors (NO, CO, and O₂) are selected for pressure sensors 404-3, 404-4, and 404-5 (model numbers NO-D4, CO-D4, and O₂-G2; AlphaSense Ltd.; Essex, United Kingdom). To correctly operate the NO and CO sensors, a potentiostatic circuit is built to control the chemical sensor, and a transimpedence amplifier is used to convert the current generated from the NO and CO sensors to a measureable voltage. The O₂ sensor does not require a potentiostatic circuit, and the signal can be obtained by using a transimpedence amplifier to convert the current generated by the sensor into a measureable voltage.

We now describe exemplary designs of microcontroller 110 of portable device 100 in FIG. 1. As mentioned previously, one main function of microcontroller 110 is to receive analog sensor signals from the set of sensors in the physical flow chamber device and transmit digitized sensor signals to a mobile device, such as a personal tablet or smartphone, using either wired or wireless connection. In one embodiment, portable device 100 employs a USB host shield to enable microcontroller 110 to communicate with mobile device 118 through a USB connection.

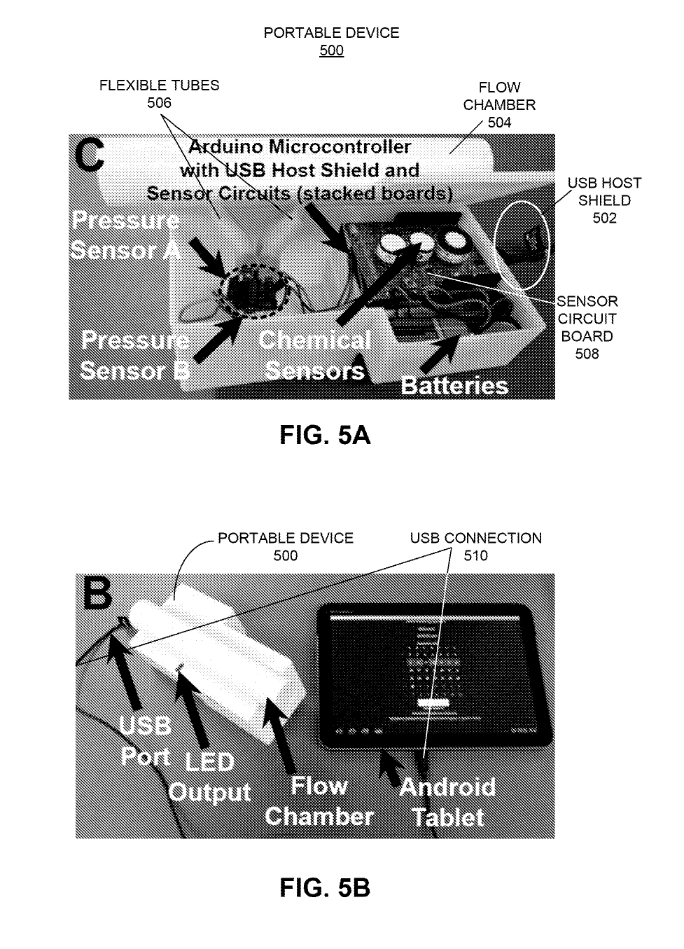

FIG. 5A illustrates an exemplary design of portable device 100 (referred to hereafter as portable device 500) and the associated microcontroller 110 in FIG. 1 in accordance with an embodiment of the present disclosure. In portable device 500, a microcontroller (Arduino Uno, R2; Strambino, Italy), which is not directly visible, is paired with a USB host shield 502 (SparkFun Electronics, DEV-09947; Boulder, Colo.) and then integrated into portable device 500 to control the external sensors (i.e., pressure sensors A and B on the left, and three chemical sensors on the right) and transmit sensor signals to a mobile device (not shown). Note that pressure sensors A and B are connected to pressure taps (invisible from the figure) in flow chamber 504 through two flexible tubes 506.

In the embodiment of FIG. 5A, the Arduino microcontroller is implemented on a separate circuit board, which is stacked underneath the visible sensor circuit board 508 located at the lower right-hand side of portable device 500. In some embodiments, however, microcontroller 110 is integrated with sensor circuits 108 on the same circuit board. In one embodiment, the microcontroller in portable device 500 is equipped with a 10-bit resolution analog-to-digital converter (ADC), which adequately digitizes analog sensor signals from all of the two pressure sensors and three chemical sensors. The Arduino microcontroller system is also configured to transmit the digitalized sensor signals from the microcontroller to the mobile device through USB host shield 502.

The following is an exemplary process of two breath maneuvers controlled by the aforementioned Arduino microcontroller. During the first breath maneuver, the Arduino microcontroller collects data from the chemical sensors first at a rate of 100 samples/sec for a total of 15 sec (data from each chemical sensor is recorded for five seconds in a sequential manner). In a second breath maneuver for spirometry testing, data points are collected from pressure sensors A and B at a rate of 50 samples/sec. The microcontroller rapidly alternates between each pressure sensor to replicate a real-time collection of samples and occurs for a total of 18 sec, allowing ample time for the patient to perform the sequential breath maneuvers.

FIG. 5B illustrates the coupling between portable device 500 and a mobile device in accordance with an embodiment of the present disclosure. As can be seen in FIG. 5B, portable device 500 is coupled to a mobile device, in this case an Android tablet, through a USB connection 510.

We now describe the concept of the software application, which can be used to control the operation of portable device 100 in FIG. 1. In one embodiment, the software application is installed on mobile device 118 in FIG. 1. The application may be written in Java programming language or any other programming language that is supported by mobile device 118. Note that the application provides an interface between user 112 and portable device 100.

FIG. 6 presents a flowchart illustrating a process 600 for using portable device 100 to monitor common lung function parameters and/or biomarkers of the exhaled breath in accordance with an embodiment of the present disclosure. During operation, the application starts by initializing microcontroller 110 (step 602). This initialization step can include, but is not limited to: setting the data rate for the microcontroller, defining variables, and naming mobile device accessory. Next, the application displays instructions on mobile device 118 for user 112 to properly perform breath maneuvers required by portable device 100 (step 604). Note that prior to step 604, the application may also display welcome screen on mobile device 118 to user 112.

Next, the application signals user 112 to begin exhaling into flow chamber 104 (step 606). In one embodiment, the application uses LED lights to signal the user when to begin breathing into the flow chamber for data collection. The application then receives digitized sensor signals from microcontroller 110 through an interface, such as a USB connection (step 608). Note that these digitized sensor signals can include both pressure/flow sensor signals and chemical biomarker sensor signals. The application then signals user 112 to stop exhaling into flow chamber 104 (step 610). In one embodiment, the application uses LED lights to signal the user when to stop breathing into the flow chamber for data collection.

In some embodiments of process 600 there are additional or fewer operations. Moreover, the order of the operations may be changed, and/or two or more operations may be combined into a single operation.

Note that to perform the two breath maneuvers required to complete a lung function test using portable device 100, steps 606-610 may be repeated twice. For example, for the first breath maneuver, the application instructs the user to perform 15 seconds of tidal breathing to collect exhaled biomarker data. Hence, the time interval between signaling user 112 to begin exhaling into flow chamber 104 and signaling user 112 to stop exhaling into flow chamber 104 can be set to 15 seconds. The user may then be instructed to take a predetermined rest period (e.g, 5 seconds) before the application signals the user to perform the second breath maneuver, i.e., a full spirometry breath maneuver. During the second breath maneuver, the application returns to step 604 and instructs the user to exhale for at least 6 sec to obtain a suitable FVC alternate and an acceptable spirometry maneuver. In the second maneuver, the time interval between signaling user 112 to begin exhaling into flow chamber 104 and signaling user 112 to stop exhaling into flow chamber 104 can be set to at least 6 seconds. Note that PEF values are also extracted from the second breath maneuver.

In some embodiments, after receiving the digitized sensor signals from microcontroller 110 at mobile device 118, each data point received is decoded and interpreted using linear regression equations determined from a set of subsequent calibration experiments. All data received from chemical sensors and pressure/flow sensors, including time, date, and Global Positioning System (GPS) location, are written and saved in a comma-separated value (.csv) file. Next, the average NO, CO, and O₂ concentrations, PEF, instantaneous flow rate, and the spirometry graph are shown to the user on the mobile device display. These data can be optionally emailed to the user's health care provider using the native operating system email application on the mobile device.

In one embodiment, to create a spirometry graph that maps exhaled breath flow rates against exhaled lung volume after each spirometry maneuver, flow rate measurements from each pressure/flow sensor are recorded in separate arrays. More specifically, after the maneuver has been completed, data from both pressure/flow sensors are merged in a systematic manner so that a better representation of the true exhaled flow rates can be achieved. A blank array is created, which will be referred to as the merged array, and the application compares the values of the two pressure/flow sensors at each point in time. In the example of FIG. 5A, if the flow rate of pressure sensor A is less than 100 L/min, then the flow rate measured by pressure sensor B is written to the merged array. If the flow rate of pressure sensor A is greater than 100 L/min, then the flow rate measured by pressure sensor A is added to the merged array. This results in a merged array that combines the accuracy of pressure sensor B at low flow rates with the ability of pressure sensor A to measure peak flow rates, creating a dataset that better reflects the actual exhaled breath flow rates from a patient—at both the high end and low end of their normal flow rate ranges.

In some embodiments, data from each chemical sensor received from the microcontroller is also converted to a concentration value when it is received by the software application. A numerical array is created for each chemical biomarker and all data received after the time constant found for each sensor is averaged to obtain an estimate of the concentration of the three chemical biomarkers in exhaled breath.

The present disclosure provides concept and specific design examples of a portable device which allows patients to monitor both common lung function parameters (FVC, PEF, FEV, FEV₁, etc.) and exhaled breath biomarkers using their personal mobile device at any time and any location. The proposed portable device combines mechanisms for PEF, spirometry, and exhaled breath biomarker measurements into a single device, making lung function monitoring less time-consuming and easier to obtain. The current invention will allow for more comprehensive data assembly for an individualized medicine approach and will enable long-distance monitoring and diagnostics by a physician even when the patient has limited access to a medical facility. Furthermore, increased monitoring of pulmonary and, potentially, other functions will aid in assembling databases that will further promote understanding of the clinical presentation of various diseases and may allow for earlier disease diagnostics and intervention. The flow metering function of the portable device is performed using an obstruction flow meter equipped with two differential pressure sensors, each focusing on half of the expected volume flow rate range. This design is preferred due to its simplicity in construction and its lack of intricate parts, which allows it to be easily cleaned and maintained.

The foregoing description is intended to enable any person skilled in the art to make and use the disclosure, and is provided in the context of a particular application and its requirements. Moreover, the foregoing descriptions of embodiments of the present disclosure have been presented for purposes of illustration and description only. They are not intended to be exhaustive or to limit the present disclosure to the forms disclosed. Accordingly, many modifications and variations will be apparent to practitioners skilled in the art, and the general principles defined herein may be applied to other embodiments and applications without departing from the spirit and scope of the present disclosure. Additionally, the discussion of the preceding embodiments is not intended to limit the present disclosure. Thus, the present disclosure is not intended to be limited to the embodiments shown, but is to be accorded the widest scope consistent with the principles and features disclosed herein.

1. A personal lung function monitoring device, comprising:
a physical measuring device, which further comprises:
a flow chamber configured to receive a flow of exhaled breath; and
a set of sensors integrated with the flow chamber, wherein the set of sensors is configured to measure a set of properties of the exhaled breath; and
a microcontroller coupled to the physical measuring device and configured to receive sensor signals from the set of sensors and transmit processed sensor signals to a mobile device.

2. The personal lung function monitoring device of claim 1, wherein the set of properties of the exhaled breath includes one or more common lung function parameters and/or one or more biomarkers of the exhaled breath.

3. The personal lung function monitoring device of claim 1, wherein the set of sensors includes at least one sensor to measure a common lung function parameter and at least one sensor to measure a biomarker of the exhaled breath

4. The personal lung function monitoring device of claim 1, wherein the set of sensors includes at least one flow sensor and at least one chemical sensor.

5. The personal lung function monitoring device of claim 1, wherein the set of sensors includes one or more sensors configured to perform a spirometry test on the exhaled breath.

6. The personal lung function monitoring device of claim 1, wherein the set of sensors includes one or more sensors configured to perform a peak expiratory flow (PEF) measurement on the exhaled breath.

7. The personal lung function monitoring device of claim 1, wherein the set of sensors includes two or more chemical sensors configured to measure two or more biomarkers of the exhaled breath.

8. The personal lung function monitoring device of claim 7, wherein the two or more biomarkers include two or more of the following:
nitric oxide (NO);
carbon monoxide (CO);
oxygen (O₂); and
other chemical biomarkers.

9. The personal lung function monitoring device of claim 1, wherein the set of sensors includes:
one or more sensors for spirometry tests of the exhaled breath;
one or more sensors for PEF measurements of the exhaled breath; and
one or more sensors for biomarker measurements of the exhaled breath.

10. The personal lung function monitoring device of claim 1, wherein the set of sensors is arranged linearly along the path of the exhaled breath through the flow chamber.

11. The personal lung function monitoring device of claim 1, wherein the flow chamber includes a cylindrical tube which has a first end and a second end,
wherein the exhaled breath enters the cylindrical tube from the first end and exits the cylindrical tube from the second end; and
wherein the wall of the cylindrical tube includes a set of openings configured to receive the set of sensors

12. The personal lung function monitoring device of claim 11, wherein a given opening in the set of openings is configured so that the size and shape of the given opening match the size and shape of a given sensor in the set of sensors, which is inserted into the given opening.

13. The personal lung function monitoring device of claim 11, wherein the cylindrical tube is partitioned into a first section and a second section by an orifice plate placed in the path of the exhaled breath, wherein the hole of the orifice plate has a diameter that is smaller than the diameter of the cylindrical tube.

14. The personal lung function monitoring device of claim 13, wherein the set of sensors includes a differential pressure sensor, which is connected to two pressure taps: the first pressure tap is positioned in the first section before the orifice plate, and the second pressure tap is positioned in the second section of the cylindrical tube after the orifice plate.

15. The personal lung function monitoring device of claim 14, wherein the set of sensors includes a second differential pressure sensor which is used cooperatively with the differential pressure sensor to perform a spirometry test.

16. The personal lung function monitoring device of claim 14, wherein the set of sensors further includes a set of chemical sensors, which is positioned in the second section of the cylindrical tube and is further away from the orifice plate than the second pressure tap.

17. The personal lung function monitoring device of claim 1, wherein the microcontroller includes an analog-to-digital converter (ADC) configured to digitize the received sensor signals.

18. The personal lung function monitoring device of claim 1, wherein the physical measuring device further comprises sensor circuits associated with the set of sensors.

19-20. (canceled)

21. A physical measuring device for personal lung function monitoring, comprising:
a flow chamber configured to receive a flow of exhaled breath; and
a set of sensors integrated with the flow chamber, wherein the set
of sensors is configured to measure a set of properties of the exhaled breath.

22-36. (canceled)

37. A method for performing a lung function test using a personal lung function monitoring device, the method comprising:
receiving exhaled breath from a user at a physical measuring device of the personal lung function monitoring device, which comprises:
a flow chamber configured to receive the exhaled breath; and
a set of sensors integrated with the flow chamber and configured to measure a set of properties of the exhaled breath;
processing analog sensor signals from the set of sensors at a microcontroller of the personal lung function monitoring device coupled to the physical measuring device; and
transmitting the processed sensor signals from the microcontroller to a mobile device coupled with the personal lung function monitoring device
